(19) United States
(12) Patent Application Publication
(10) Pub. No.: US 2019/0174999 A1
(43) Pub. Date: Jun. 13, 2019

(54) ENDOSCOPE AND ENDOSCOPE SYSTEM

(71) Applicant: Olympus Corporation, Tokyo (JP)

(72) Inventor: Fuminori Wake, Tokyo (JP)

(73) Assignee: Olympus Corporation, Tokyo (JP)

(21) Appl. No.: 16/274,607

(22) Filed: Feb. 13, 2019

Related U.S. Application Data
(63) Continuation of application No. PCT/JP2017/024438, filed on Jul. 4, 2017.

(30) Foreign Application Priority Data
Aug. 19, 2016 (JP) 2016161051

(51) Int. Cl. A61B 1/015; A61B 1/00; A61B 1/12

(52) U.S. Cl. CPC A61B 1/015; A61B 1/00119; A61B 1/00096; A61B 1/126; A61B 1/00091

(57) ABSTRACT

The disclosed technology is directed to an endoscope comprises an operation portion. An insertion portion having respective proximal and distal ends. The proximal end is configured to be attached to the operation portion and the distal end is used to be inserted into a subject. The distal end of the insertion portion includes a distal surface having at least an observation window, a channel opening, and an elongated feed conduit having a slanted feed passage all which formed therein to communicate simultaneously with the operation portion and a procedure instrument during an operation on the subject. The elongated feed conduit having the slanted feed passage used to direct fluid into the subject. The slanted passage feed includes an ejection port that ejects the fluid from the slanted feed passage at an angle to generate a vortex flow so that mucus or mist-form particles are diffused away from the observation window.

### CROSS-REFERENCE TO RELATED APPLICATIONS

This application is a continuation application of PCT Application No. PCT/JP2017/024438 filed on Jul. 4, 2017, which in turn claim priority to the Japanese Patent Application No. 2016-161051 filed on Aug. 19, 2016 in Japan which is hereby incorporated by reference in its entirety.

### TECHNICAL FIELD

The disclosed technology relates to an endoscope and an endoscope system with which procedure based on ablation is carried out under endoscopic observation.

### DESCRIPTION OF THE RELATED ART

In recent years, in the medical field, endoscopic submucosal dissection (hereinafter, referred to as ESD) has been carried out as early cancer treatment. This ESD is a procedure of inserting a high-frequency electric knife in an endoscope and endoscopically removing submucosa of an early cancer. In the ESD, incision, coagulation, or the like of tissue is carried out based on ablation by use of the high-frequency electric knife.

At this time, an endoscopic image often becomes unclear due to evaporation of mucus and fat in a lumen and adhesion of the fat and so forth that have become particles (smoke) in a mist form to an observation lens of the endoscope or filling of the inside of the lumen with the particles in the mist form. For this reason, in operative procedure such as the ESD, a countermeasure for ensuring favorable visibility against the smoke or the like generated by the ablation procedure is necessary.

Here, for example, the following technique is disclosed in Japanese Patent Laid-open No. 2005-176908. Specifically, a fluid curtain starting-end-side flow path is disposed on the upper side of an observation window disposed at the distal end of an insertion portion of a rigid endoscope (laparoscope) and a fluid curtain terminating-end-side flow path is disposed on the lower side. Furthermore, contamination of the observation window due to smoke or the like generated by ablation procedure of an affected portion or the like is prevented by causing a fluid of carbon dioxide or the like to flow from a fluid curtain starting-end-side opening toward a fluid curtain terminating-end-side opening and forming a fluid curtain.

However, it is difficult to apply the technique disclosed in Japanese Patent Laid-open No. 2005-176908 described hereinbefore to operative procedure such as an ESD of a digestive organ as it is although the technique is effective to operative procedure carried out in a comparatively-wide space (in the abdominal cavity), such as laparoscopic surgery.

Specifically, in operative procedure such as an ESD in which a high-frequency electric knife or the like is protruded from a procedure instrument channel disposed near an observation window and ablation or the like is carried out in a narrow space (lumen) of a digestive organ such as a large intestine, the observation window is often disposed at a position extremely close to an affected portion. In such a case, for example, there is a possibility that the tissue of the treatment target undulates and gets deformed due to the wind pressure of the fluid curtain and it becomes difficult to cause the procedure instrument or the like to approach. Furthermore, if particles in a mist form are generated near the observation window, there is a possibility that these particles are blown onto the observation window by the fluid curtain and it becomes difficult to ensure favorable visibility. Moreover, there is a possibility that mucus or the like in the lumen is caused to foam due to the wind pressure of the fluid curtain and it becomes difficult to ensure favorable visibility.

The disclosed technology is made in view of the circumstances described hereinbefore and intends to provide an endoscope and an endoscope system that can ensure favorable visibility even when procedure is carried out near an observation window.

### BRIEF SUMMARY OF EMBODIMENTS

The disclosed technology is directed to an endoscope comprises an operation portion. An insertion portion having respective proximal and distal ends. The proximal end is configured to be attached to the operation portion and the distal end is used to be inserted into a subject. The distal end of the insertion portion includes a distal surface having at least an observation window, a channel opening, and an elongated feed conduit having a slanted feed passage all which being formed therein to communicate simultaneously with the operation portion and a procedure instrument during an operation on the subject. The observation window is used to observe inside of the subject via an imaging unit. The channel opening is used to direct the procedure instrument therethrough to perform the operation on the subject. The elongated feed conduit having the slanted feed passage used to direct fluid into the subject during the operation. The slanted passage feed includes an ejection port that ejects the fluid from the slanted feed passage at an angle to generate a vortex flow so that mucus or mist-form particles are diffused away from the observation window and to ensure clear visibility.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a schematic configuration diagram of an endoscope system.

FIG. 2 is an end view of an insertion portion.

FIG. 3 is a sectional view of a major portion along line in FIG. 2.

FIG. 4 is an explanatory diagram depicting the ejection direction of a gas in the end view of the insertion portion.

FIG. 5 is an explanatory diagram depicting the ejection direction of the gas in a sectional view along line V-V in FIG. 4.

FIG. 6 is an explanatory diagram depicting the behavior of the gas ejected in a lumen.

FIG. 7 relates to a first modification example and is an end view of the insertion portion.

FIG. 8 relates to the first modification example and is a sectional view of a major portion along line VIII-VIII in FIG. 7.

FIG. 9 relates to a second modification example and is an end view of the insertion portion.

FIG. 10 relates to the second modification example and is a sectional view of a major portion along line X-X in FIG. 9.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

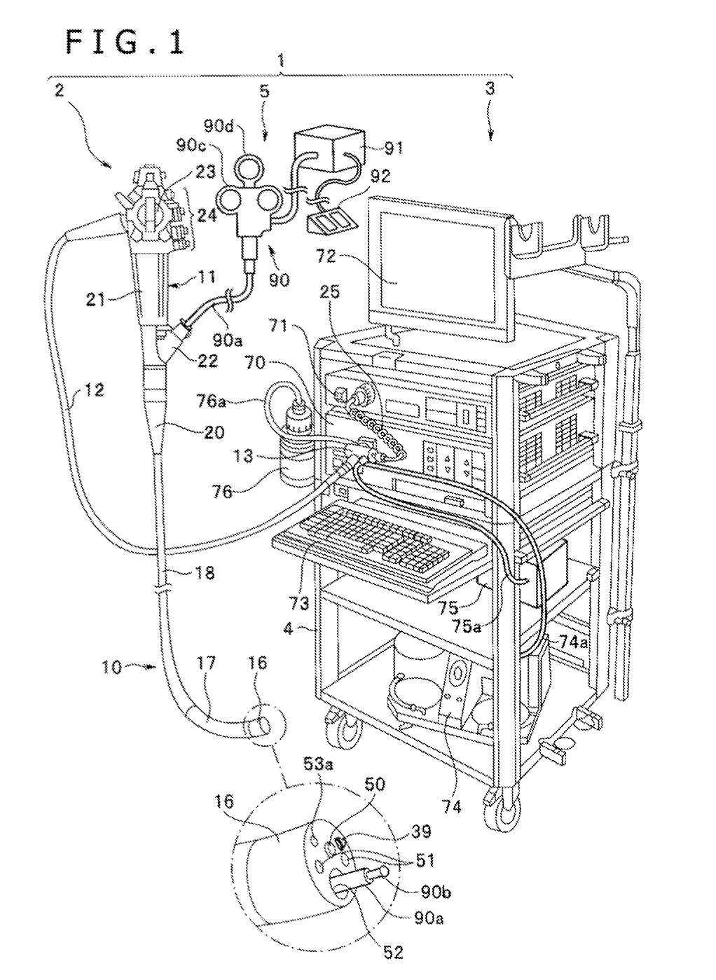

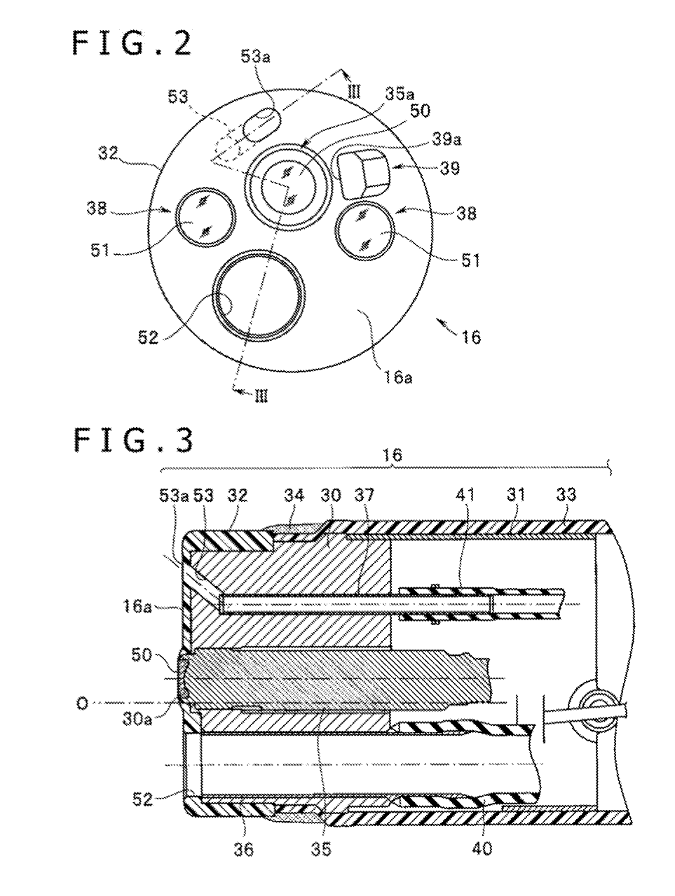

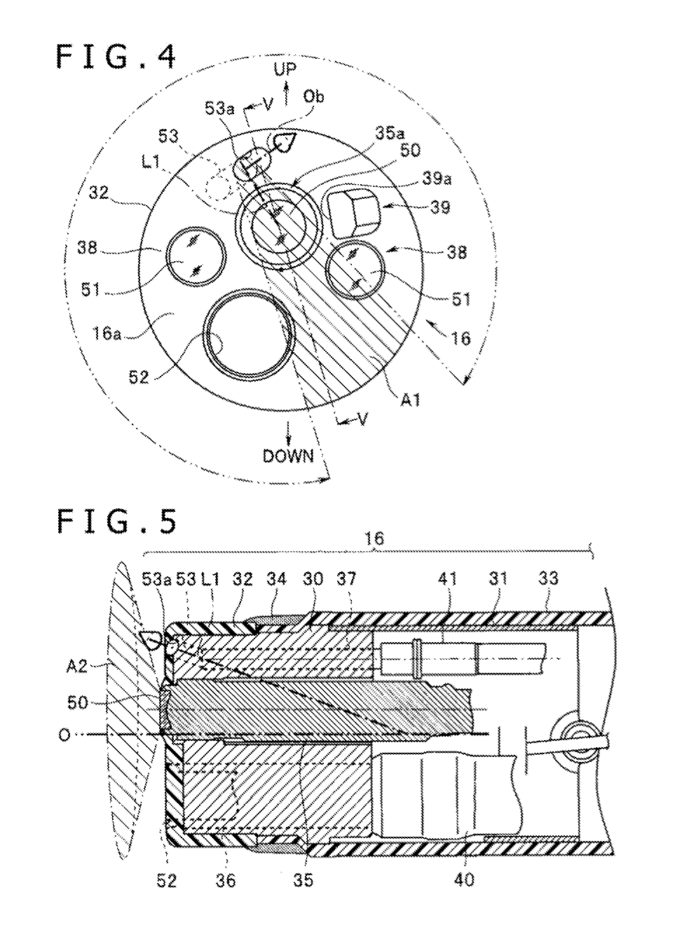

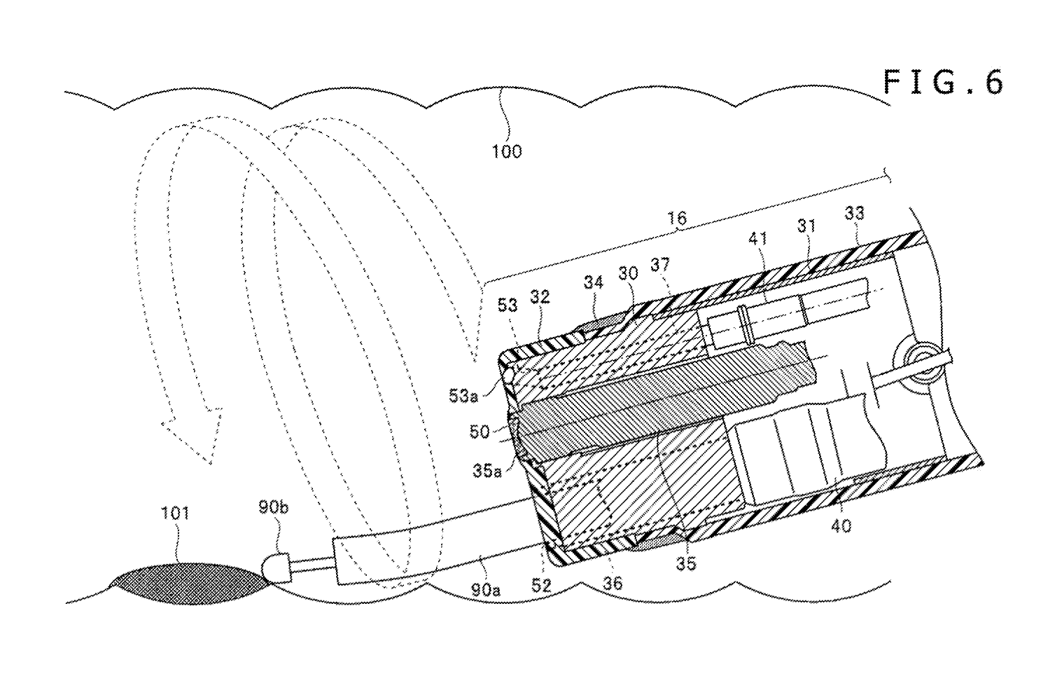

A mode of the present disclosure will be described below with reference to the drawings. The drawings relate to one embodiment of the present disclosure. FIG. 1 is a schematic configuration diagram of an endoscope system. FIG. 2 is an end view of an insertion portion. FIG. 3 is a sectional view of a major portion along line in FIG. 2. FIG. 4 is an explanatory diagram depicting the ejection direction of a gas in the end view of the insertion portion. FIG. 5 is an explanatory diagram depicting the ejection direction of the gas in a sectional view along line V-V in FIG. 4. FIG. 6 is an explanatory diagram depicting the behavior of the gas ejected in a lumen.

An endoscope system 1 depicted in FIG. 1 is configured to have an endoscope 2, equipment 3 for an endoscope mounted on a movable trolley 4, and ablation apparatus 5 as a procedure instrument for ablation.

Here, the equipment 3 for an endoscope are configured to have light source apparatus 70 that generates illumination light with which an observation target region is irradiated, a video processor 71 that executes predetermined image processing on a video signal obtained by imaging, a monitor 72 that displays the video signal as an observed image, a keyboard 73 that is an input portion for inputting of various kinds of commands and data by a user, a suction unit 74 that sucks a liquid and a gas, and a pump unit 75 for gas feed.

Furthermore, a bottle 76 that stores a cleaning liquid that is a liquid used for cleaning and so forth is detachably attached to a support column of the trolley 4. To this bottle 76, a built-in pump unit for gas feed (not depicted) incorporated in the light source apparatus 70 or the like separately from the pump unit 75 for gas feed described hereinbefore is connected. Furthermore, the built-in pump unit for gas feed can discharge the cleaning liquid in the bottle 76 by supplying compressed air.

The ablation apparatus 5 is configured to have a high-frequency knife 90 as a procedure instrument that gives energy to tissue in a subject to carry out ablation, a high-frequency power supply 91 that provides power to this high-frequency knife 90, and a foot switch 92 to carry out turning-on/off of the power provided from the high-frequency power supply 91 to the high-frequency knife 90, and so forth.

The high-frequency knife 90 is configured to have an elongated sheath 90a, a procedure portion 90b that protrudes from the distal end of the sheath, an operation portion 90d disposed continuously with the proximal end of the sheath 90a, and a slider 90c for operation that can advance and retreat relative to the operation portion 90d.

The endoscope 2 is an endoscope for a digestive organ for example and is an endoscope for the large intestine as one further example thereof. This endoscope 2 is configured to have an elongated insertion portion 10 inserted into a subject, an operation portion 11 disposed continuously with the proximal end of the insertion portion 10 in the longitudinal direction, a universal cord 12 extended from the operation portion 11, and a connector 13 disposed at the extension end of the universal cord 12.

The insertion portion 10 is configured to have a distal portion 16 located at the most distal end, a bending portion 17 disposed continuously with the proximal side of this distal portion 16, and an elongated flexible tube portion 18 disposed continuously with the proximal side of this bending portion 17.

The operation portion 11 has an operation portion main unit 21 disposed continuously with the proximal side of the flexible tube portion 18 with the intermediary of a breaking prevention portion 20. In this operation portion main unit 21, for example, a procedure instrument insertion port 22 through which various kinds of procedure instruments and so forth can be inserted, a bending operation knob 23 for causing the bending portion 17 to make bending action in the upward-downward and right-left directions, and switches 24 for carrying out operation of various kinds of endoscope functions are disposed.

The universal cord 12 is formed of a composite cord inside which various kinds of cables and tubes extended from the side of the operation portion 11 are inserted. Here, inside the universal cord 12 in the present embodiment, for example, a light guide formed of an optical fiber, plural signal lines that transmit a video signal and so forth, a supply passage of gas and liquid (gas-feed/liquid-feed channel as a cleaning fluid conduit) and a discharge passage that are formed of tubes, a gas feed channel disposed separately from the gas-feed/liquid-feed channel (neither is depicted) are inserted as the various kinds of cables and tubes.

The connector 13 is configured to be freely attached and detached to and from the light source apparatus 70. Furthermore, by connecting this connector 13 to the light source apparatus 70, the light guide inserted in the universal cord 12 can be optically connected to the light source apparatus 70 and the gas-feed/liquid-feed channel inserted in the universal cord 12 can be connected to the built-in pump for gas feed.

Furthermore, a connection cable 25 configured to be freely attached and detached to and from the video processor 71 is extended from the connector 13. In addition, by connecting this connection cable 25 to the video processor 71, the signal lines inserted in the universal cord 12 can be electrically connected to the video processor 71.

Moreover, in the connector 13, ferrules (neither is depicted) that can be each connected to a tube 74a extended from the suction unit 74, a tube 75a extended from the pump unit 75 for gas feed, and a tube 76a extended from the bottle 76 are disposed. Furthermore, by connecting these respective tubes 74a, 75a, and 76a to the connector 13, the discharge passage in the universal cord 12 can be connected to the suction unit 74 and the gas feed channel in the universal cord 12 can be connected to the pump unit 75 for gas feed. In addition, the gas-feed/liquid-feed channel in the universal cord 12 can be connected to the bottle 76.

Next, with reference to FIG. 2 and FIG. 3, the configuration of the distal portion 16 of the endoscope 2 will be described in detail.

As depicted in FIG. 3, inside the distal portion 16, a distal end forming portion 30 formed of a hard resin, metal, or the like is disposed.

To the proximal-side outer circumference of this distal end forming portion 30, a bending piece located at the most distal end (most distal bending piece 31) in a bending piece set disposed in the bending portion 17 is fitted.

Furthermore, on the distal side of the distal end forming portion 30, a distal cover 32 that covers the distal surface and the distal-side outer circumferential surface of this distal end forming portion 30 is disposed. In addition, a distal surface 16a of the distal portion 16 is formed by this distal cover 32.

Moreover, the proximal-side outer circumference of the distal end forming portion 30 is covered by bending rubber 33 that extends from the bending portion 17. The distal portion of this bending rubber 33 is firmly fixed to the outer circumferential portion of the distal end forming portion 30 by a spool bonding portion 34 in the state of being disposed continuously with the proximal end of the distal cover 32.

As depicted in FIG. 3, the following components are each fitted into the distal end forming portion 30: an imaging unit 35 as an observation member for observing a subject; e.g. two light guide bundle holding pipes (not depicted) as illuminating members that emit illumination light for illuminating the subject; a procedure instrument channel connecting pipe 36; a cleaning tube connecting pipe (not depicted) that feeds a liquid such as a cleaning liquid to clean an observation window 50 to be described hereinafter; and a gas feed tube connecting pipe 37.

The imaging unit 35 is configured to have an observation lens 35a at the most distal end. This observation lens 35a is an optical member for forming an objective lens unit at the distal end of the imaging unit 35 and is exposed to the outside from a hole formed in the distal cover 32. Furthermore, the observation window 50 is formed in the distal surface 16a by the observation lens 35a exposed from the distal cover 32 in this manner (see FIG. 2).

The distal portion of a light guide bundle (not depicted) that can be optically connected to the light source apparatus 70 via the connector 13 is held by each light guide bundle holding pipe. Moreover, illumination lenses 38 are optically connected to the distal side of the light guide bundle holding pipes. These illumination lenses 38 are exposed to the outside from holes formed in the distal cover 32. Furthermore, illumination windows 51 are formed in the distal surface 16a by the illumination lenses 38 exposed in this manner (see FIG. 2).

The distal portion of a procedure instrument channel 40 that is a soft tube body is connected to the proximal portion of the procedure instrument channel connecting pipe 36. In the operation portion 11, with the proximal side of this procedure instrument channel 40, the procedure instrument insertion port 22 is made to communicate and the discharge passage that can be connected to the suction unit 74 via the connector 13 is made to communicate.

Meanwhile, a channel opening 52 as an opening formed in the distal cover 32 is made to communicate with the distal portion of the procedure instrument channel 40. This allows the distal side of the high-frequency knife 90 inserted in the procedure instrument insertion port 22 to protrude into a subject through the channel opening 52. Furthermore, the suction unit 74 can suck fluids such as gases and body fluids in the subject through the channel opening 52.

The gas-feed/liquid-feed channel that can be connected to the bottle 76 and the built-in pump for gas feed in the light source apparatus 70 via the connector 13 is connected to the proximal portion of the cleaning tube connecting pipe.

Meanwhile, a cleaning nozzle 39 (see FIG. 2) as a cleaning fluid ejection portion is made to communicate with the distal side of the cleaning tube connecting pipe. An ejection port 39a of this cleaning nozzle 39 is opened toward the observation window 50. This allows the cleaning nozzle 39 to eject, as a cleaning fluid, a gas directly supplied from the built-in pump for gas feed or a liquid such as a cleaning liquid discharged from the bottle 76 by the gas from the built-in pump for gas feed toward the observation window 50.

A gas feed channel 41 as a conduit that can be connected to the pump unit 75 for gas feed via the connector 13 is connected to the proximal portion of the gas feed tube connecting pipe 37.

Meanwhile, a gas feed passage 53 formed in the distal end forming portion 30 and the distal cover 32 are made to communicate with the distal side of the gas feed tube connecting pipe 37.

For this gas feed passage 53, for example as depicted in FIGS. 4 and 5, a center axis Ob of an ejection port 53a is set in such a manner that a gas (for example carbon dioxide) from the gas feed channel 41 is ejected in a direction that is a direction intersecting a longitudinal axis O of the insertion portion 10 and is oriented toward a position other than the position at which the observation window 50 is disposed in the distal surface 16a.

To put it concretely, for the gas feed passage 53, the ejection direction of the gas (that is, the direction of the center axis Ob of the ejection port 53a, the ejection center direction when the gas is ejected with a certain angle range) is set to a direction that is such a direction that the gas ejected from the ejection port 53a does not directly traverse the observation window 50 (that is, direction that does not traverse an area A1 (see FIG. 4) surrounded by tangents to the ejection port 53a and the observation window 50) and does not traverse a field-of-view area A2 (see FIG. 5) of the observation window 50.

More specifically, the center axis Ob of the ejection port 53a is set in such a manner that the gas is ejected in a direction that intersects each of the longitudinal axis O of the insertion portion 10 and the plane orthogonal to the longitudinal axis and is such a direction as to get further away from the distal surface 16a.

In this case, for example, as depicted in FIG. 4, if the upward/downward directions (UP/DOWN directions) of the distal surface 16a are defined as substantially the same directions as the upward/downward directions of an image captured through the observation window based on the upward/downward directions of this image, it is desirable for the ejection port 53a to be disposed at a position on the upper side relative to the observation window 50.

Furthermore, for example, as depicted in FIG. 4, it is preferable that, for the ejection port 53a, the ejection direction of the gas from the conduit when the distal surface 16a is seen in plan view (direction of the center axis Ob) be set to a direction obtained by shifting in a rotational direction around an axis along the longitudinal axis O of the insertion portion 10 with respect to a straight line L1 that couples the ejection port 53a and the observation window 50 in the distal surface 16a. In particular, it is preferable that the direction of the center axis Ob of the ejection port 53a be set to a direction substantially orthogonal to the straight line L1.

The observation window 50 is often disposed at substantially the center of the distal surface 16a in the endoscope 2. In such an endoscope 2, it is preferable that the direction of the center axis Ob of the ejection port 53a when the distal surface 16a is seen in plan view be a direction along a tangent direction of the outer circumference of the distal surface 16a furthermore.

Furthermore, it is desirable that the ejection port 53a be set at such a position as to get further away from the channel opening 52 across the observation window 50, and it is preferable that the ejection direction of the gas from the ejection port 53a (center axis Ob) be set to such a direction as to get further away from the channel opening 52.

With such a configuration, for example, as depicted in FIG. 6, when ablation of a lesion portion 101 generated in a lumen of a large intestine or the like is carried out by the procedure portion 90b of the high-frequency knife 90, a gas of carbon dioxide or the like pressure-fed from the pump unit 75 for gas feed is ejected from the ejection port 53a into the lumen.

The gas ejected from this ejection port 53a is oriented in a direction that is a direction intersecting the longitudinal axis O of the insertion portion 10 and is toward a position other than the position at which the observation window 50 is disposed in the distal surface 16a. Thus, the gas is made to impinge on a body wall 100 without directly traversing the observation window 50.

Then, the gas that has impinged on the body wall 100 travels toward the front side of the distal portion 16 while being guided by this body wall 100 to thereby generate a transverse vortex flow (swirl flow) around the longitudinal axis O in the lumen (see dashed lines in FIG. 6).

Due to this transverse vortex flow, a gentle gas flow is generated in a space on the front side relative to the distal portion 16. Therefore, even if mucus and fat evaporate into a mist form due to the ablation by the procedure portion 90b, this mist-form fat and so forth are diffused (diluted) to a wide range without remaining at one place.

In particular, if the ejection port 53a has a configuration that ejects the gas in such a manner that a direction that intersects each of the longitudinal axis O of the insertion portion 10 and the plane orthogonal to the longitudinal axis and is such a direction as to get further away from the distal surface 16a is employed as the center axis Ob, the transverse vortex flow described hereinbefore can be generated more surely and the mist-form fat and so forth can be diffused (diluted) to a wide range more appropriately.

According to such an embodiment, because the endoscope 2 has the gas feed channel 41 as the conduit that feeds a gas from the outside of a subject to the distal side of the insertion portion 10 and the ejection port 53a that ejects the gas from the gas feed channel 41 in a direction that is a direction intersecting the longitudinal axis O of the insertion portion 10 and is oriented toward a position other than the position at which the observation window 50 is disposed in the distal surface 16a, favorable visibility can be ensured also when procedure is carried out near the observation window 50.

Specifically, by ejecting the gas from the ejection port 53a in the direction toward a position other than the position at which the observation window 50 is disposed, contamination and so forth due to blowing of particles of body fluid and fat that evaporate into a mist form near the observation window 50 onto the observation window 50 by the gas from the ejection port 53a can be prevented and favorable visibility of the observation window 50 can be ensured.

Furthermore, in a comparatively-narrow lumen such as a large intestine, even when the gas is ejected from the ejection port 53a toward a position other than the position at which the observation window 50 is disposed, this gas is guided by the body wall 100 and generates a transverse vortex flow, and a gas flow is generated by this transverse vortex flow. Thereby, particles of fat and so forth that evaporate into a mist form on the front side of the observation window 50 can be appropriately diffused without being caused to remain at one place and favorable visibility can be ensured.

In this case, by disposing the ejection port 53a on the upper side relative to the observation window 50 (upward direction of the distal surface 16a defined based on the upward/downward directions of an image captured through the observation window) and at a position spaced apart from the channel opening 52 across the observation window 50 and setting the ejection direction of the gas from the ejection port 53a (center axis Ob) to such a direction as to get further away from the channel opening 52, the gas ejected from the ejection port 53a can be prevented from being directly blown onto biological tissue, body fluid, and so forth near the channel opening 52. Therefore, it is possible to appropriately prevent the occurrence of undulation of the biological tissue, foaming of the body fluid or the like, and so forth due to the gas ejected from the ejection port 53a in the vicinity of the lesion portion 101 which the procedure portion 90b of the high-frequency knife 90 protruded from the channel opening 52 approaches.

Furthermore, because the ejection port 53a is disposed on the upper side relative to the observation window 50, it is possible to appropriately prevent the occurrence of a situation in which a liquid such as water that accumulates on the lower side in the lumen is raised and the liquid comes into view when the gas is ejected.

In this case, the transverse vortex flow around the longitudinal axis O can be generated more favorably by setting the ejection direction of the gas from the ejection port 53a (direction of the center axis Ob) to a direction substantially orthogonal to the straight line L1 that couples the ejection port 53a and the observation window 50 in the distal surface 16a (more preferably, the direction in plan view of the distal surface 16a is a direction substantially along a tangent to the outer circumference of this distal surface 16a).

Moreover, more favorable visibility can be ensured by introducing gas, body fluid, and so forth containing particles generated in the subject into the procedure instrument channel 40, which is another conduit, through the channel opening 52, which is an opening, and causing the gas, body fluid, and so forth to flow out to the outside of the subject by suction by the suction unit 74. Here, for example, if the procedure instrument channel 40 is a conduit with a diameter of 3.2 mm, by using the high-frequency knife 90 whose diameter of the sheath 90a is about 2.4 mm, suction can be carried out through the gap between the procedure instrument channel 40 and the high-frequency knife 90 simultaneously with procedure such as ablation, with the high-frequency knife 90 remaining inserted in the procedure instrument channel 40.

Furthermore, by disposing the cleaning nozzle 39 for ejecting a cleaning fluid such as a cleaning liquid toward the observation window 50 separately from the ejection port 53a, the observation window 50 can be cleaned according to need and more favorable visibility can be ensured.

That is, in such an embodiment, the ejection port 53a that ejects a gas in a direction toward a position other than the position at which the observation window 50 is disposed and the cleaning nozzle 39 that ejects the cleaning fluid toward the observation window 50 are different from each other in the purpose and the structure (technical idea).

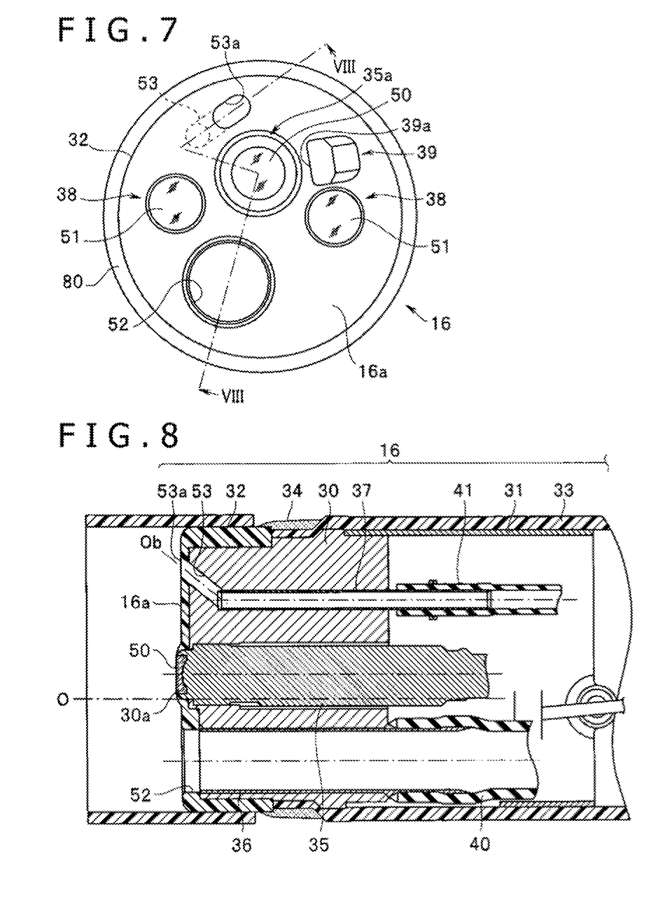

Here, for example, as depicted in FIGS. 7 and 8, it is also possible to attach a circular cylindrical hood 80 that protrudes in the direction of the longitudinal axis O from the distal surface 16a to the outer circumferential edge of the distal portion 16.

In this case, the ejection port 53a is set to eject a gas in a direction toward a position other than the position at which the observation window 50 is disposed and toward the inner circumferential surface of the hood 80. At this time, to generate a transverse vortex flow more surely, it is more preferable that the ejection port 53a be set to eject the gas in a direction substantially along a tangent to the outer circumference of the distal surface 16a in the directions in plan view of the distal surface 16a.

According to such a configuration, the transverse vortex flow can be appropriately generated and mist-form particles and so forth can be diffused not only in a large intestine or the like but also in a narrow lumen such as a stomach. In particular, by setting the ejection direction of the gas from the ejection port 53a to the direction substantially along a tangent to the outer circumference of the distal surface 16a in plan view, the ejected gas can be appropriately moved along the inner circumferential surface of the hood 80, so that a favorable transverse vortex flow can be generated and mist-form particles and so forth can be diffused more appropriately.

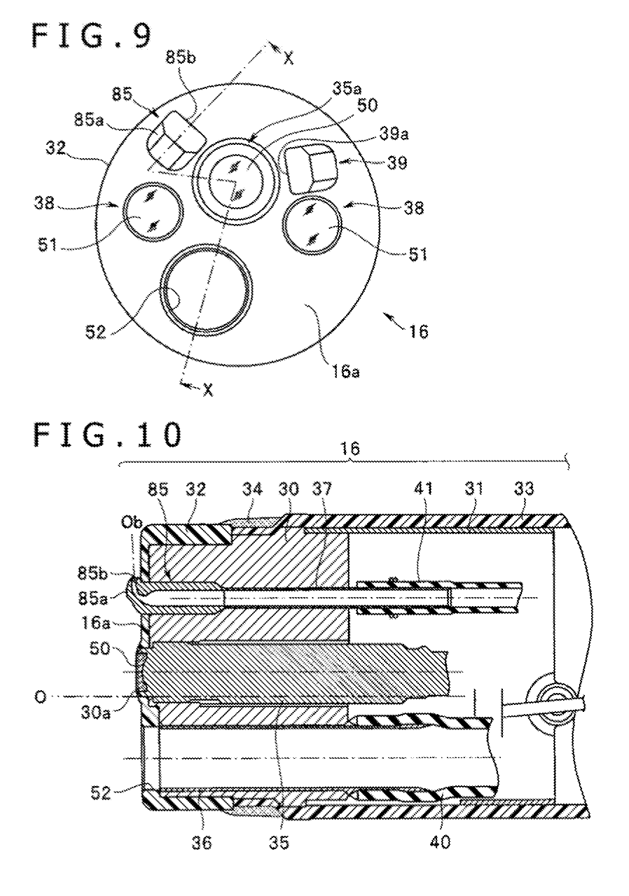

Furthermore, for example, as depicted in FIGS. 9 and 10, it is also possible to dispose a nozzle 85 continuously with the distal side of the gas feed tube connecting pipe 37 and set an opening of a nozzle head 85a that protrudes from the distal surface 16a as an ejection port 85b.

If the configuration is made in this manner, the direction of the ejection port 85b can be arbitrarily set according to the use purpose and so forth of the endoscope 2 in a range that satisfies a predetermined condition that the direction is a direction intersecting the longitudinal axis O and is a direction toward a position other than the position at which the observation window 50 is disposed in the distal surface 16a, or the like. Moreover, in the embodiment described hereinbefore, a gas may be sucked from the inside of a subject (lumen) through the channel opening 52 or the like while the amount of gas and the pressure in the subject (lumen) are adjusted to prevent the pressure in the subject (lumen) from rising up to a prescribed pressure or higher due to the amount of ejection of the gas from the ejection port 53a into the subject (lumen).

Furthermore, in this case, for employing e.g. a configuration in which, for the gas that is sucked from the inside of the subject (lumen) and is caused to flow out to the outside of the subject, a mist and so forth are removed and then the resulting gas is used as the gas to be ejected into the subject (lumen) again, the suction unit 74 and the pump unit 75 for gas feed may be connected to be used as a return current unit that circulates the gas between the inside and outside of the subject (lumen).

In sum, one aspect of the disclosed technology is directed to an endoscope comprises an operation portion. An insertion portion having respective proximal and distal ends. The proximal end is configured to be attached to the operation portion and the distal end is used to be inserted into a subject. The distal end of the insertion portion includes a distal surface having at least an observation window, a channel opening, and an elongated feed conduit having a slanted feed passage all which being formed therein to communicate simultaneously with the operation portion and a procedure instrument during an operation on the subject. The observation window is used to observe inside of the subject via an imaging unit. The channel opening is used to direct the procedure instrument therethrough to perform the operation on the subject. The elongated feed conduit having the slanted feed passage used to direct fluid into the subject during the operation. The slanted passage feed includes an ejection port that ejects the fluid from the slanted feed passage at an angle to generate a vortex flow so that mucus or mist-form particles are diffused away from the observation window and to ensure clear visibility.

The ejection port is configured to eject the fluid in a direction that is intersecting a longitudinal axis of the insertion portion. The direction is also directed toward a position other than a position at which the observation window is disposed in the distal surface. The direction is also directed toward a position other than a position at which the channel opening is disposed. A second passage communicates with the channel opening so as to suck a fluid in the subject from the inside of the subject through the channel opening by a suction unit. The ejection port ejects the fluid such as gas in a manner that a direction that is a direction intersecting a longitudinal axis of the insertion portion and is toward a position other than the position at which the observation window is disposed in the distal surface is employed as a center direction of the ejection of the gas. The ejection port ejects the gas in a direction that intersects each of the longitudinal axis and a plane orthogonal to the longitudinal axis and is such a direction as to get further away from the distal surface. The endoscope further comprises a cleaning fluid conduit that feeds a cleaning fluid from the outside of the subject to the distal side of the insertion portion. A cleaning fluid ejection portion that is disposed in the distal surface and ejects the cleaning fluid toward the observation window. The elongated feed conduit and the ejection port are disposed separately from the cleaning fluid conduit and the cleaning fluid ejection portion. The endoscope further comprises another conduit that has a second channel opening disposed on the distal side of the insertion portion and causes a gas containing particles generated in the subject to flow out to the outside of the subject through the second channel opening. For the ejection port, an ejection direction of the gas from the elongated feed conduit when the distal surface is seen in plan view is set to a direction obtained by shifting in a rotational direction around an axis along the longitudinal axis of the insertion portion with respect to a straight line that couples the ejection port and the observation window in the distal surface. For the ejection port, an ejection direction of the gas from the elongated feed conduit when the distal surface is seen in plan view is set to a direction substantially orthogonal to a straight line that couples the ejection port and the observation window in the distal surface. For the ejection port, an ejection direction of the gas from the elongated feed conduit when the distal surface is seen in plan view is set to a direction along a tangent to an outer circumferential edge of the distal surface. The ejection port is disposed at a position on an upper side relative to the observation window if upward and downward directions of the distal surface are defined based on upward and downward directions of an image captured through the observation window. The ejection port is disposed at a position on the upper side relative to the channel opening if the upward and downward directions of the distal surface are defined based on the upward and downward directions of the image captured through the observation window. A hood that protrudes in a direction of the longitudinal axis from the distal surface is attached to an outer circumferential edge of the distal surface. The ejection port ejects the gas toward an inner circumferential surface of the hood. The ejection port is formed in a nozzle which proximal side communicates with the elongated feed conduit and which distal side protrudes from the distal surface.

Another aspect of the disclosed technology is directed to an endoscope system comprises an operation portion and an insertion portion having respective proximal and distal ends. The proximal end is configured to be attached to the operation portion and the distal end is used to be inserted into a subject. The distal end of the insertion portion includes a distal surface having at least an observation window, a channel opening, and an elongated feed conduit having a slanted feed passage all which being formed therein to communicate simultaneously with the operation portion and a procedure instrument during an operation on the subject. The observation window being used to observe inside of the subject via an imaging unit. The channel opening is used to direct the procedure instrument therethrough to perform the operation on the subject. The elongated feed conduit having the slanted feed passage used to direct fluid into the subject during the operation. The slanted passage feed includes an ejection port that ejects the fluid from the slanted feed passage at an angle to generate a vortex flow so that mucus or mist-form particles are diffused away from the observation window and to ensure clear visibility. A pump that supplies a fluid to the elongated feed conduit and a procedure instrument for ablation having a procedure portion protruded from the distal surface.

A further aspect of the disclosed technology is directed to an endoscope comprises an operation portion and a procedure instrument is detachably attached to the operation portion. An elongated insertion portion having respective proximal and distal ends. The proximal end is configured to be attached to the operation portion and the distal end is used to be inserted into a subject during a treatment. The elongated insertion portion having at least an observation window, a channel opening, and an elongated feed conduit having an slanted feed passage all which being concealed along the elongated insertion portion and their respective ends being exposed at a distal face of the insertion portion to communicate simultaneously with the operation portion and the procedure instrument during the treatment of the subject. The slanted feed passage is used to direct fluid into the subject during the treatment and the slanted passage feed includes an ejection port that ejects the fluid from the distal face at an angle to generate a vortex flow so that mucus or mist-form particles are diffused away from the observation window and to ensure clear visibility during the treatment.

The insertion portion further includes an imaging unit that permits a user to transfer an image of the subject using a pair of illumination windows. The endoscope system further comprises a movable trolley configured to support various equipments such as a light source apparatus, a video processor unit, an image processing unit, a monitor, a suction unit, and the pump all of which communicate with one another during the operation on the subject. An endoscope is configured to be attached to the various equipments during the operation on the subject. The endoscope comprises the operation portion and the procedure instrument being detachably attached to the operation portion. The insertion portion having at least the observation window, the channel opening, and the elongated feed conduit all which being concealed along the elongated insertion portion and their respective ends being exposed at a distal face of the insertion portion to communicate simultaneously with the operation portion and the procedure instrument during the treatment of the subject.

The present invention is not limited to the embodiment described hereinbefore. Various modifications and changes are possible and they also fall within the technical scope of the present invention. For example, in the embodiment and respective modification examples described hereinbefore, one example in which the present invention is applied to the endoscope 2 for the large intestine is mainly described. However, it is obvious that the present invention is not limited thereto and can be applied also to other endoscopes such as a digestive organ endoscope for another region like a stomach or esophagus. Furthermore, it is obvious that the configurations of the embodiment and modification examples described hereinbefore may be combined as appropriate.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An endoscope comprising:
an operation portion; and
an insertion portion having respective proximal and distal ends wherein the proximal end configured to be attached to the operation portion and the distal end being used to be inserted into a subject, the distal end of the insertion portion includes a distal surface having at least an observation window, a channel opening, and an elongated feed conduit having a slanted feed passage all which being formed therein to communicate simultaneously with the operation portion and a procedure instrument during an operation on the subject and wherein
the observation window being used to observe inside of the subject via an imaging unit;
the channel opening being used to direct the procedure instrument therethrough to perform the operation on the subject;
the elongated feed conduit having the slanted feed passage used to direct fluid into the subject during the operation and wherein the slanted passage feed includes an ejection port that ejects the fluid from the slanted feed passage at an angle to generate a vortex flow so that mucus or mist-form particles are diffused away from the observation window and to ensure clear visibility.

2. The endoscope of claim 1, wherein the ejection port is configured to eject the fluid in a direction that
intersecting a longitudinal axis of the insertion portion,
being directed toward a position other than a position at which the observation window is disposed in the distal surface, and
being directed toward a position other than a position at which the channel opening is disposed.

3. The endoscope of claim 1, wherein a second passage communicates with the channel opening so as to suck a fluid in the subject from the inside of the subject through the channel opening by a suction unit.

4. The endoscope of claim 1, wherein the ejection port ejects the fluid such as gas in a manner that a direction that is a direction intersecting a longitudinal axis of the insertion portion and is toward a position other than the position at which the observation window is disposed in the distal surface is employed as a center direction of the ejection of the gas.

5. The endoscope of claim 1, wherein the ejection port ejects the gas in a direction that intersects each of the longitudinal axis and a plane orthogonal to the longitudinal axis and is such a direction as to get further away from the distal surface.

6. The endoscope of claim 1, further comprising
a cleaning fluid conduit that feeds a cleaning fluid from the outside of the subject to the distal side of the insertion portion, and
a cleaning fluid ejection portion that is disposed in the distal surface and ejects the cleaning fluid toward the observation window, wherein
the elongated feed conduit and the ejection port are disposed separately from the cleaning fluid conduit and the cleaning fluid ejection portion.

7. The endoscope of claim 1 further comprises another conduit that has a second channel opening disposed on the distal side of the insertion portion and causes a gas containing particles generated in the subject to flow out to the outside of the subject through the second channel opening.

8. The endoscope of claim 1, wherein, for the ejection port, an ejection direction of the gas from the elongated feed conduit when the distal surface is seen in plan view is set to a direction obtained by shifting in a rotational direction around an axis along the longitudinal axis of the insertion portion with respect to a straight line that couples the ejection port and the observation window in the distal surface.

9. The endoscope of claim 1, wherein, for the ejection port, an ejection direction of the gas from the elongated feed conduit when the distal surface is seen in plan view is set to a direction substantially orthogonal to a straight line that couples the ejection port and the observation window in the distal surface.

10. The endoscope of claim 1, wherein, for the ejection port, an ejection direction of the gas from the elongated feed conduit when the distal surface is seen in plan view is set to a direction along a tangent to an outer circumferential edge of the distal surface.

11. The endoscope of claim 1, wherein the ejection port is disposed at a position on an upper side relative to the observation window if upward and downward directions of the distal surface are defined based on upward and downward directions of an image captured through the observation window.

12. The endoscope of claim 10, wherein the ejection port is disposed at a position on the upper side relative to the channel opening if the upward and downward directions of the distal surface are defined based on the upward and downward directions of the image captured through the observation window.

13. The endoscope of claim 1, wherein
a hood that protrudes in a direction of the longitudinal axis from the distal surface is attached to an outer circumferential edge of the distal surface, and
the ejection port ejects the gas toward an inner circumferential surface of the hood.

14. The endoscope of claim 1, wherein the ejection port is formed in a nozzle which proximal side communicates with the elongated feed conduit and which the distal side protrudes from the distal surface.

15. An endoscope system comprising:
an operation portion;
an insertion portion having respective proximal and distal ends wherein the proximal end configured to be attached to the operation portion and the distal end being used to be inserted into a subject, the distal end of the insertion portion includes a distal surface having at least an observation window, a channel opening, and an elongated feed conduit having a slanted feed passage all which being formed therein to communicate simultaneously with the operation portion and a procedure instrument during an operation on the subject, wherein
the observation window being used to observe inside of the subject via an imaging unit;
the channel opening being used to direct the procedure instrument therethrough to perform the operation on the subject;
the elongated feed conduit having the slanted feed passage used to direct fluid into the subject during the operation and wherein the slanted passage feed includes an ejection port that ejects the fluid from the slanted feed passage at an angle to generate a vortex flow so that mucus or mist-form particles are diffused away from the observation window and to ensure clear visibility;
a pump that supplies a fluid to the elongated feed conduit; and
a procedure instrument for ablation having a procedure portion protruded from the distal surface.

16. An endoscope comprising:
an operation portion;
a procedure instrument being detachably attached to the operation portion; and
an elongated insertion portion having respective proximal and distal ends wherein the proximal end configured to be attached to the operation portion and the distal end being used to be inserted into a subject during a treatment, the elongated insertion portion having at least an observation window, a channel opening, and an elongated feed conduit having an slanted feed passage all which being concealed along the elongated insertion portion and their respective ends being exposed at a distal face of the insertion portion to communicate simultaneously with the operation portion and the procedure instrument during the treatment of the subject and wherein the slanted feed passage being used to direct fluid into the subject during the treatment and wherein
the slanted passage feed includes
an ejection port that ejects the fluid from the distal face at an angle to generate a vortex flow so that mucus or mist-form particles are diffused away from the observation window and to ensure clear visibility during the treatment.

17. The endoscope of claim 16, wherein the insertion portion further includes an imaging unit that permits a user to transfer an image of the subject using a pair of illumination windows.

18. The endoscope system of claim 15, further comprising:
a movable trolley configured to support various equipments such as a light source apparatus, a video processor unit, an image processing unit, a monitor, a suction unit, and the pump all of which communicate with one another during the operation on the subject; and
an endoscope configured to be attached to the various equipment during the operation on the subject, the endoscope comprising
the operation portion;
the procedure instrument being detachably attached to the operation portion; and
the insertion portion having at least the observation window, the channel opening, and the elongated feed conduit all which being concealed along the elongated insertion portion and their respective ends being exposed at a distal face of the insertion portion to communicate simultaneously with the operation portion and the procedure instrument during the treatment of the subject.
